(19) United States
(12) Patent Application Publication
(10) Pub. No.: US 2017/0354645 A1
(43) Pub. Date: Dec. 14, 2017

(54) METHODS FOR TREATING KIDNEY DISORDERS

(71) Applicant: Eli Lilly and Company, Indianapolis, IN (US)

(72) Inventors: Asim Bikash DEY, Carmel, IN (US); Michael James GENIN, Danville, IN (US); Laura Frey MICHAEL, Indianapolis, IN (US)

(21) Appl. No.: 15/524,332

(22) PCT Filed: Nov. 24, 2015
(86) PCT No.: PCT/US2015/062369

§ 371 Date: May 4, 2017

Related U.S. Application Data
(60) Provisional application No. 62/086,278, filed on Dec. 2, 2014.

(51) Int. Cl. A61K 31/422; A61K 9/00

(52) U.S. Cl. CPC A61K 31/422; A61K 9/0053

(57) ABSTRACT

Provided are methods and formulations for treating kidney disorders in man and animals, which include the use of a compound of the Formula I and pharmaceutically acceptable salts thereof.

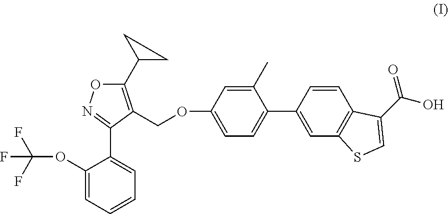 [SMILES: Cc1cc(OCc2c(-c3ccccc3OC(F)(F)F)noc2C2CC2)ccc1-c1ccc2c(C(=O)O)csc2c1]

Renal or kidney disorders in man and animals involve an alteration in the normal physiology and function of the kidney. Renal disorders can result from a wide range of acute and chronic conditions and events, including physical, chemical, or biological injury, insult or trauma, disease, and various inflammatory and autoimmune diseases. Kidney disorders can lead to reduced kidney function, seriously compromising quality and duration of life. Regardless of the initial insult or cause, kidney disorders are characterized by progressive destruction of the renal parenchyma and the loss of functional nephrons. This progression often leads to chronic kidney disease (CKD) and end-stage renal disease and failure (ESRD/ESRF).

CKD is characterized by the progressive loss of kidney function. Increased albuminuria and gradual, progressive loss of renal function are primary manifestations in CKD. Decreased renal function results in increased blood creatinine and blood urea nitrogen (BUN). CKD patients experience over time an increase in albuminuria, proteinuria, serum creatinine, and renal histopathological lesions.

In humans, CKD has been, and continues to be, a considerable social and economic problem in all industrialized countries. In the USA, 102,567 patients began dialysis in 2003 (341 patients/year per million), and similar rates were found in developing countries and in particular ethnic groups (2006, USRDS Am J Kidney Dis 47:1-286; Meguid El Nahas, A., and Bello, A. K. 2005. Chronic kidney disease: the global challenge. Lancet 365:331-340.). However, these numbers are a small fraction of the millions of patients who are thought to have some degree of renal impairment. In the United States, the prevalence of chronically reduced kidney function is estimated to be around 10% of adults (http://kidney.niddk.nih.gov/kudiseases/pubs/kustats/index.htm, pages 1-4). Worsening CKD evolves into ERSD for many patients, requiring either dialysis or kidney transplant. Glomerular filtration rate (GFR) is used to classify the severity of CKD for patients, with lower GFR corresponding to more severe CKD. Reducing the rate at which GFR declines in patients is expected to delay or prevent the development of ESRD. Angiotensin converting enzyme inhibitors or angiotensin II receptor antagonists are used as current standard of care to slow the progression of CKD to ERSD, but these have been shown inadequate to stop the ultimate progression to dialysis.

The prevalence of renal disorders is also high in cats, whereas chronic renal failure is considered the most important one. The prevalence of feline CKD has been reported to reach up to 20%, with 53% of those cats being older than 7 years (Lefebre, Toutain 2004, J. Vet. Pharm. Therap. 27, 265-281; Wolf, North. Am. Vet Congress 2006). Survival in cats with mild to moderate azotemia and extrarenal clinical signs (International Renal Interest Society (IRIS) stages 2 and 3) ranges from 1 to 3 years. Current therapy aims to delay the progression of the disease in cats by improving renal function. This includes dietary protein restriction, modification of dietary lipid intake, phosphate restriction and treatment with angiotensin-converting enzyme (ACE) inhibitors (P. J. Barber (2004) The Kidney, in: Chandler E A, Gaskell C J, Gaskell R M, (eds.) Feline Medicine and Therapeutics, 3rd edition, Blackwell Publishing, Oxford, UK).

U.S. Pat. No. 7,863,302 is directed to compounds which are described as farnesoid X receptor (FXR) modulators. The compounds of U.S. Pat. No. 7,863,302 are described as being useful for treating dyslipidemia and related diseases.

There remains a need in the art to provide alternative therapies for treating kidney disorders in man and animals. Particular unmet needs are alternative therapies for treating human and feline CKD. The present inventions provides alternative methods and formulations for treating kidney disorders, including CKD, in man and animals, using the compound of Formula I

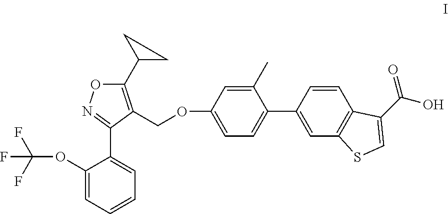 [SMILES: Cc1cc(OCc2c(-c3ccccc3OC(F)(F)F)noc2C2CC2)ccc1-c1ccc2c(C(=O)O)csc2c1]

(6-{4-[5-Cyclopropyl-3-(2-trifluoromethoxy-phenyl)-isoxazol-4-ylmethoxy]-2-methyl-phenyl}-benzo[b]thiophene-3-carboxylic acid) or a pharmaceutically acceptable salt thereof. The compound of Formula I has an activity profile which indicates it has a utility in the treatment of kidney disorders in man and animal.

In an embodiment of the invention, provided are methods of treating a kidney disorder in a patient in need thereof, comprising administering a compound of the Formula I, or a pharmaceutically acceptable salt thereof. In an embodiment of the invention, provided is a compound of Formula I, or a pharmaceutically acceptable salt thereof, for use in the treatment of a kidney disorder. In an embodiment of the invention, provided is the use of a compound of Formula I, or a pharmaceutically acceptable salt thereof, for the manufacture of a medicament for treating a kidney disorder. In an embodiment of the invention, provided is the use of a compound of Formula I, or a pharmaceutically acceptable salt thereof, for the treatment of a kidney disorder. In an embodiment of the invention, provided are pharmaceutical formulations for treating a kidney disorder in a patient comprising a compound of the Formula I, or a pharmaceutically acceptable salt thereof, and one or more pharmaceutically acceptable carriers. In an embodiment of the invention, provided is a unit dose formulation for treating a kidney disorder in a patient comprising a compound of Formula I, or a pharmaceutically acceptable salt thereof, and one or more pharmaceutically acceptable carriers.

In an embodiment of the invention, provided are methods of decreasing proteinuria in a patient in need thereof, comprising administering a compound of the Formula I, or a pharmaceutically acceptable salt thereof. In an embodiment of the invention, provided is a compound of Formula I, or a pharmaceutically acceptable salt thereof, for use in decreasing proteinuria. In an embodiment of the invention, provided is the use of a compound of Formula I, or a pharmaceutically acceptable salt thereof, for the manufacture of a medicament for decreasing proteinuria. In an embodiment of the invention, provided is the use of a compound of Formula I, or a pharmaceutically acceptable salt thereof, for decreasing proteinuria. In an embodiment of the invention, provided are pharmaceutical formulations for decreasing proteinuria in a patient comprising a compound of the Formula I, or a pharmaceutically acceptable salt thereof, and one or more pharmaceutically acceptable carriers. In an embodiment of the invention, provided is a unit dose formulation for decreasing proteinuria in a patient comprising a compound of Formula I, or a pharmaceutically acceptable salt thereof, and one or more pharmaceutically acceptable carriers.

In an embodiment of the invention, provided are methods of decreasing albuminuria in a patient in need thereof, comprising administering a compound of the Formula I, or a pharmaceutically acceptable salt thereof. In an embodiment of the invention, provided is a compound of Formula I, or a pharmaceutically acceptable salt thereof, for use in decreasing albuminuria. In an embodiment of the invention, provided is the use of a compound of Formula I, or a pharmaceutically acceptable salt thereof, for the manufacture of a medicament for decreasing albuminuria. In an embodiment of the invention, provided is the use of a compound of Formula I, or a pharmaceutically acceptable salt thereof, for decreasing albuminuria. In an embodiment of the invention, provided are pharmaceutical formulations for decreasing albuminuria in a patient comprising a compound of the Formula I, or a pharmaceutically acceptable salt thereof, and one or more pharmaceutically acceptable carriers. In an embodiment of the invention, provided is a unit dose formulation for decreasing albuminuria in a patient comprising a compound of Formula I, or a pharmaceutically acceptable salt thereof, and one or more pharmaceutically acceptable carriers.

In an embodiment of the invention, provided are methods of decreasing the loss of glomerular filtration rate (GFR) reflected by an increase in serum creatinine in a patient in need thereof, comprising administering a compound of the Formula I, or a pharmaceutically acceptable salt thereof. In an embodiment of the invention, provided is a compound of Formula I, or a pharmaceutically acceptable salt thereof, for use in decreasing the loss of GFR reflected by an increase in serum creatinine. In an embodiment of the invention, provided is the use of a compound of Formula I, or a pharmaceutically acceptable salt thereof, for the manufacture of a medicament for decreasing the loss of GFR reflected by an increase in serum creatinine. In an embodiment of the invention, provided is the use of a compound of Formula I, or a pharmaceutically acceptable salt thereof, for decreasing the loss of GFR reflected by an increase in serum creatinine. In an embodiment of the invention, provided are pharmaceutical formulations for decreasing the loss of GFR reflected by an increase in serum creatinine in a patient comprising a compound of the Formula I, or a pharmaceutically acceptable salt thereof, and one or more pharmaceutically acceptable carriers. In an embodiment of the invention, provided is a unit dose formulation for decreasing the loss of GFR reflected by an increase in serum creatinine in a patient comprising a compound of Formula I, or a pharmaceutically acceptable salt thereof, and one or more pharmaceutically acceptable carriers.

In an embodiment of the invention, provided are methods of slowing the rate of progression to ESRD in a patient in a patient in need thereof, comprising administering a compound of the Formula I, or a pharmaceutically acceptable salt thereof. In an embodiment of the invention, provided is a compound of Formula I, or a pharmaceutically acceptable salt thereof, for use in slowing the rate of progression to ESRD in a patient. In an embodiment of the invention, provided is the use of a compound of Formula I, or a pharmaceutically acceptable salt thereof, for the manufacture of a medicament for slowing the rate of progression to ESRD. In an embodiment of the invention, provided is the use of a compound of Formula I, or a pharmaceutically acceptable salt thereof, for slowing the rate of progression to ESRD. In an embodiment of the invention, provided are pharmaceutical formulations for slowing the rate of progression to ESRD in a patient comprising a compound of the Formula I, or a pharmaceutically acceptable salt thereof, and one or more pharmaceutically acceptable carriers. In an embodiment of the invention, provided is a unit dose formulation for slowing the rate of progression to ESRD in a patient comprising a compound of Formula I, or a pharmaceutically acceptable salt thereof, and one or more pharmaceutically acceptable carriers.

In an embodiment of the invention, provided are methods of protecting against renal histopathological lesions in a patient in need thereof, comprising administering a compound of the Formula I, or a pharmaceutically acceptable salt thereof. In an embodiment of the invention, provided is a compound of Formula I, or a pharmaceutically acceptable salt thereof, for use in protecting against renal histopathological lesions. In an embodiment of the invention, provided is the use of a compound of Formula I, or a pharmaceutically acceptable salt thereof, for the manufacture of a medicament for protecting against renal histopathological lesions. In an embodiment of the invention, provided is the use of a compound of Formula I, or a pharmaceutically acceptable salt thereof, for protecting against renal histopathological lesions. In an embodiment of the invention, provided are pharmaceutical formulations for protecting against renal histopathological lesions in a patient comprising a compound of the Formula I, or a pharmaceutically acceptable salt thereof, and one or more pharmaceutically acceptable carriers. In an embodiment of the invention, provided is a unit dose formulation for protecting against renal histopathological lesions in a patient comprising a compound of Formula I, or a pharmaceutically acceptable salt thereof, and one or more pharmaceutically acceptable carriers.

In any of the embodiments of the invention, the patient may be a human. In any of the embodiments of the invention, the patient may be a cat. In any of the embodiments of the invention, the administration may be oral administration. In any of the embodiments of the invention, the administration may be carried out in a tablet, capsule, solution, or suspension. In any of the embodiments of the invention, the kidney disease may be glomerulonephritis, interstitial nephritis, tubulo-interstitial nephritis, pyelonephritis, lupus nephritis, chronic kidney disease, diabetic nephropathy, focal segmental glomerulosclerosis, reflux nephropathy, glumerulonephrosis, or polycystic renal disease. In any of the embodiments of the invention, the kidney disorder may be chronic kidney disease. In any of the embodiments of the invention, the compound of Formula I, or a pharmaceutically acceptable salt thereof, may be administered in an amount from about 0.1 to about 500 mg per kg weight of said patient. In any of the embodiments of the invention, the pharmaceutical formulation may be in an oral dosage form. In any of the embodiments of the invention, the pharmaceutical formulation may be a tablet, capsule, solution, or suspension. In any of the embodiments of the invention, the pharmaceutical formulation may comprise the compound of Formula I, or a pharmaceutically acceptable salt thereof, in an amount from about 0.1 to about 500 mg per kg weight of said patient. In any of the embodiments of the invention, the standard of care may be administered in separate, simultaneous, or sequential combination with the compound of Formula I. The standard of care includes, but is not limited to, angiotensin converting enzyme (ACE) inhibitors or angiotensin II receptor (ARB) antagonists.

The present invention provides the compound of Formula I, or a pharmaceutically acceptable salt thereof, which is believed to cause a reduction in proteinuria with a concomitant reduction in disease progression in patients. Further, the present invention provides the compound of Formula I, or a pharmaceutically acceptable salt thereof, which is believed to be effective in the treatment of a kidney disorder in patients. Further, the present invention provides the compound of Formula I, or a pharmaceutically acceptable salt thereof, which is believed to be effective in the treatment of chronic kidney disease in patients. The present invention provides the compound of Formula I, or a pharmaceutically acceptable salt thereof, which is believed to cause a reduction in albuminuria with a concomitant reduction in disease progression in patients. The present invention provides the compound of Formula I, or a pharmaceutically acceptable salt thereof, which is believed to cause a reduction in serum creatinine with a concomitant reduction in disease progression in patients.

The term “kidney disorder” means any renal disorder, renal disease, or kidney disease where there is any alteration in normal physiology and function of the kidney. This can result from a wide range of acute and chronic conditions and events, including physical, chemical or biological injury, insult, trauma or disease, such as for example hypertension, diabetes, congestive heart failure, lupus, sickle cell anemia and various inflammatory, infectious and autoimmune diseases, HIV(or related diseases)-associated nephropathies etc. This term includes but is not limited to diseases and conditions such as kidney transplant, nephropathy; CKD; glomerulonephritis; inherited diseases such as polycystic kidney disease; nephromegaly (extreme hypertrophy of one or both kidneys); nephrotic syndrome; ESRD; acute and chronic renal failure; interstitial disease; nephritis; sclerosis, an induration or hardening of tissues and/or vessels resulting from causes that include, for example, inflammation due to disease or injury; renal fibrosis and scarring; renal-associated proliferative disorders; and other primary or secondary nephrogenic conditions. Fibrosis associated with dialysis following kidney failure and catheter placement, e.g., peritoneal and vascular access fibrosis, is also included.

In some embodiments, the kidney disorder may be generally defined as a “nephropathy” or “nephropathies”. The terms “nephropathy” or “nephropathies” encompass all clinical-pathological changes in the kidney which may result in kidney fibrosis and/or glomerular diseases (e.g. glomerulosclerosis, glomerulonephritis) and/or chronic renal insufficiency, and can cause end stage renal disease and/or renal failure. In some embodiments, the terms “nephropathy” or “nephropathies” refers specifically to a disorder or disease where there is either the presence of proteins (i.e. proteinuria) in the urine of a subject and/or the presence of renal insufficiency.

The term “fibrosis” refers to abnormal processing of fibrous tissue, or fibroid or fibrous degeneration. Fibrosis can result from various injuries or diseases, and can often result from chronic transplant rejection relating to the transplantation of various organs. Fibrosis typically involves the abnormal production, accumulation, or deposition of extracellular matrix components, including overproduction and increased deposition of, for example, collagen and fibronectin. As used herein, the terms “kidney fibrosis” or “renal fibrosis” or “fibrosis of the kidney” refer to diseases or disorders associated with the overproduction or abnormal deposition of extracellular matrix components, particularly collagen, leading to the degradation or impairment of kidney function.

The term “patient” includes living organisms in which kidney disorders can occur, or which are susceptible to kidney disorders. The term includes humans and non-human animals including mammals (felines/cats, dogs, horses, pigs, cows, goats, sheep, rodents (mice, rats), rabbits, squirrels, bears, primates (chimpanzees, monkeys, gorillas)); birds (chickens, ducks, Peking ducks, geese); and transgenic species thereof. Preferably, the patient is a mammal. More preferably, the patient is a human or a feline.

The compound of Formula I, and salts thereof, may be made by processes known in the art or apparent to one of ordinary skill in the art. A description for a preparation of the compound of Formula I is found in U.S. Pat. No. 7,863,302, particularly as Example 101. As noted in U.S. Pat. No. 7,863,302, the compound of Formula I is known to have two crystal forms, and these crystal forms, as well as any others, are included in this invention.

The terms “treatment,” “treat,” “treating,” and the like, are meant to include slowing or reversing the progression of a disorder. These terms also include alleviating, ameliorating, attenuating, eliminating, or reducing one or more symptoms of a disorder or condition, even if the disorder or condition is not actually eliminated and even if progression of the disorder or condition is not itself slowed or reversed.

“Effective amount” means the amount of an the compound for the methods and uses of the present invention that will elicit the biological or medical response of, or desired therapeutic effect on, a tissue, system, or patient that is being sought by the researcher, medical doctor, veterinarian, or other clinician. An effective amount of the compound may vary according to factors such as the specific disease involved, the disease state, age, sex, and weight of the patient, the ability of the compound to elicit a desired response in the patient, the response of the individual patient, the particular compound administered, the mode of administration, the bioavailability characteristics of the preparation administered, the dose regimen selected, and the use of any concomitant medications. An effective amount is also one in which any toxic or detrimental effect of the compound is outweighed by the therapeutically beneficial effects. The frequency of the administration will also be dependent upon several factors, and can be a single or multiple dose administration. For use in a patient, a typical daily dose for administration of a compound of Formula I, or a salt thereof, ranges from about 0.1 to about 500 milligrams per kilogram of the patient's body weight, and more desirably, from about 1 to about 250 milligrams per kilogram of the patient's body weight, which may be administered as a single dose, or as multiple doses, in a dosing regimen which may be once or multiple times a day, week, month, or other regimen as determined by the attending diagnostician. In some instances, dosage levels below the lower limit of the aforesaid range may be more than adequate, while in other cases still larger doses may be employed.

The term “unit dose formulation” refers to a physically discrete unit suitable as unitary dosages for a patient, each unit containing a predetermined quantity of a compound of Formula I, or salt thereof, calculated to produce the desired therapeutic effect, in association with a suitable pharmaceutical carrier. In an embodiment, the compositions according to the invention are formulated in a unit dosage form, each dosage containing from about 0.1 mg to about 3500 mg, more preferably about 1 mg to about 500 mg, even more preferably about 1 mg to about 100 mg of a compound of Formula I, or salt thereof. The specification for the dosage unit forms of the invention may vary and are dictated by and directly dependent on a number of factors such as the particular therapeutic effect to be achieved, the species of the patient; its size, age, and general health; the specific disease involved; the degree or severity of the disease; the response of the individual patient; the particular compound administered; the mode of administration; the bioavailability characteristics of the preparation administered; the dose regimen selected; and the use of any concomitant medications. An oral unit dose formulation is preferred.

“Pharmaceutically acceptable” as used in this application, for example with reference to salts and formulation components such as carriers, includes “veterinarily acceptable”, and thus includes both human and non-human animal applications independently.

Salts of the compounds of the invention, including pharmaceutically acceptable salts, and common methodology for preparing them, are known in the art. See, e.g., P. Stahl, et al., HANDBOOK OF PHARMACEUTICAL SALTS: PROPERTIES, SELECTION AND USE, (VCHA/Wiley-VCH, 2002); S. M. Berge, et al., “Pharmaceutical Salts,” Journal of Pharmaceutical Sciences, Vol. 66, No. 1, January 1977.

A compound of Formula I, or a salt thereof, may be formulated as pharmaceutical compositions for administration. Such pharmaceutical compositions and processes for making the same are known in the art for both humans and non-human animals. See, e.g., REMINGTON: THE SCIENCE AND PRACTICE OF PHARMACY, (A. Gennaro, et al., eds., 19^{th} ed., Mack Publishing Co., 1995). Formulations can be administered through various means, including but not limited to oral administration; parenteral administration such as injection (intramuscular, subcutaneous, intravenous, intraperitoneal); and topical application. Oral formulations are preferred and include tablets, capsules, solutions, and suspensions. Carrier is used herein to describe any ingredient other than the active component(s) in a formulation. The choice of carrier will to a large extent depend on factors such as the particular mode of administration or application, the effect of the carrier on solubility and stability, and the nature of the dosage form.

### Efficacy Rodent Model of Chronic Renal Failure

The purpose of this assay is to analyze the compound of Formula I in a mouse chronic renal failure model, and particularly the ability to improve albuminuria and renal histopathological lesions. This assay is known as the mouse remnant kidney model, where following a unilateral nephrectomy, partial surgical removal of approximately ⅓ of the remaining kidney is carried out to replicate chronic renal failure. This model is sometimes also referred to as a “5/6^{th} nephrectomy” model that was originally developed in rat. A description of this model may be found in published US patent application US 2014/0271675 (Qi, et al.) and (Leelahavanichkul, et al. (2010) Kidney Int. 78(11): 1136-1153.

Thirty-five eight to nine week old mice (strain 129/SvEv from Taconic) are employed in the model, and all have surgeries. A sham group having five mice have surgeries where the kidneys are not removed. The mice in the remaining two groups having fifteen mice each undergo remnant surgery. The surgeries take place approximately three weeks prior to the dosing portion of the study. The dosing portion of the study is 85 days long.

The oral vehicle for testing the compound of Formula I is 5% Kolliphor® HS 15, 5% EtOH, NaCMC 1%/SLS 0.5%/Antifoam 0.05%/Povidone 0.085% from Ricca Chemical Company. The sham group is fed a conventional diet (Purina Formulab Diet 5008) and is not dosed with vehicle or the compound of Formula I during the dosing portion of the study. One of the remaining two groups is orally dosed vehicle once daily (10 ml/kg), and the other group is orally dosed once daily with the compound of Formula I at 10 mg/kg, in vehicle. Both dosing groups receive a conventional diet (Purina Formulab Diet 5008) during the dosing portion of the study.

Spot urine samples are collected on day −7 prior to the dosing portion of the study. Urine samples are taken weekly during the dosing portion of the study. Body weights are measured on days 15, 29, 43, 57, 64, 71, and 78. Blood samples are taken at week 4 of the dosing portion of the study a well as at the study termination (day 84). At week 4 of the dosing portion of the study, the mean serum concentration of the compound of Formula I 24 hours after a 10 mg/kg dose is 49±66 ng/ml.

Renal Morphology. The incidence and severity of tubular protein and glumerulosclerosis are notably reduced in the mice dosed with a compound of Formula I as compared with vehicle treatment. The incidence, but not severity, of tubular changes (dilation, atrophy) is also reduced in the mice dosed with a compound of Formula I as compared with vehicle treatment. Tubular regeneration and interstitial inflammation is mildly reduced with treatment using the compound of Formula I.

Organ Weights. Heart weights are increased in the mice with 5/6ths of their kidneys removed, regardless of treatment. This heart weight gain reflects the hypertension induced by the reduction of renal mass. Kidney weights are not different across the surgery or treatments due to hypertrophy of tubules in this remnant model.

The compound of Formula I reduces the urine ACR (albumin/creatinine ratio) in the remnant mice. In general, a reduction in the range of about 32-38% in urine ACR is observed within 2 weeks and persists through the end of the dosing portion of the study.

1. A method of treating a kidney disorder in a patient in need thereof comprising administering a compound of the formula 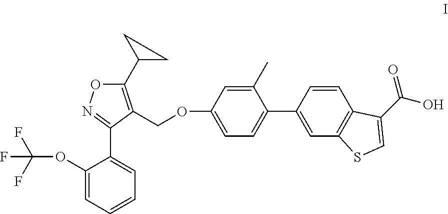 [SMILES: Cc1cc(OCc2c(-c3ccccc3OC(F)(F)F)noc2C2CC2)ccc1-c1ccc2c(C(=O)O)csc2c1]
or a pharmaceutically acceptable salt thereof.

2. The method of claim 1, wherein said patient is a human.

3. The method of claim 1, wherein said patient is a cat.

4. The method of claim 3, wherein said administration is oral administration.

5. The method of claim 4, wherein said administration is carried out using said compound in a tablet, capsule, solution, or suspension.

6. The method of claim 4, wherein said kidney disorder is glomerulonephritis, interstitial nephritis, tubulo-interstitial nephritis, pyelonephritis, lupus nephritis, chronic kidney disease, diabetic nephropathy, focal segmental glomerulosclerosis, reflux nephropathy, glumerulonephrosis, or polycystic renal disease.

7. The method of claim 6, wherein said kidney disorder is chronic kidney disease.

8-15. (canceled)

16. A pharmaceutical formulation for treating a kidney disorder in a patient comprising a compound of the formula 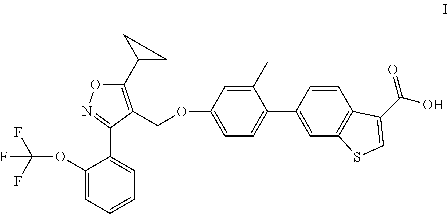 [SMILES: Cc1cc(OCc2c(-c3ccccc3OC(F)(F)F)noc2C2CC2)ccc1-c1ccc2c(C(=O)O)csc2c1]
or a pharmaceutically acceptable salt thereof, and one or more pharmaceutically acceptable carriers.

17. The pharmaceutical formulation of claim 16, wherein said pharmaceutical formulation is a unit dose formulation.

18. The pharmaceutical formulation of claim 17, wherein said pharmaceutical formulation is in an oral dosage form.

19. The pharmaceutical formulation of claim 18, wherein said pharmaceutical formulation is a tablet, capsule, solution, or suspension.

20. The pharmaceutical formulation of claim 19, wherein said kidney disorder is glomerulonephritis, interstitial nephritis, tubulo-interstitial nephritis, pyelonephritis, lupus nephritis, chronic kidney disease, diabetic nephropathy, focal segmental glomerulosclerosis, reflux nephropathy, glumerulonephrosis, or polycystic renal disease.

21. The pharmaceutical formulation of any of claim 20, wherein said kidney disorder is chronic kidney disease.

22. The method of claim 2, wherein said administration is oral administration.

23. The method of claim 22, wherein said administration is carried out using said compound in a tablet, capsule, solution, or suspension.

24. The method of claim 22, wherein said kidney disorder is glomerulonephritis, interstitial nephritis, tubulo-interstitial nephritis, pyelonephritis, lupus nephritis, chronic kidney disease, diabetic nephropathy, focal segmental glomerulosclerosis, reflux nephropathy, glumerulonephrosis, or polycystic renal disease.

25. The method of claim 24, wherein said kidney disorder is chronic kidney disease.
